(19) United States
(12) Patent Application Publication
(10) Pub. No.: US 2022/0338886 A1
(43) Pub. Date: Oct. 27, 2022

(54) SYSTEM AND METHOD TO POSITION A TRACKING SYSTEM FIELD-OF-VIEW

(71) Applicant: THINK SURGICAL, INC., Fremont, CA (US)

(72) Inventors: Daniel Patrick Bonny, Fremont, CA (US); Joel Zuhars, Fremont, CA (US)

(73) Assignee: THINK SURGICAL, INC., Fremont, CA (US)

(21) Appl. No.: 17/620,798

(22) PCT Filed: Jun. 18, 2020
(86) PCT No.: PCT/US2020/038657

§ 371 Date: Dec. 20, 2021

Related U.S. Application Data
(60) Provisional application No. 62/863,624, filed on Jun. 19, 2019.

(51) Int. Cl. A61B 17/17; A61B 34/20

(52) U.S. Cl. CPC A61B 17/1764; A61B 34/20; A61B 34/25

(57) ABSTRACT

A method and system are provided to assist in positioning the field-of-view (FOV) of an optical tracking system during a computer-assisted surgical procedure. The method includes displaying a view from a visible light detector on a display, and generating an outline as an overlay on the display of a FOV of two or more optical tracking detectors on the displayed view from the visible light detector. A user then positions at least one of: a) the two or more optical tracking detectors, or b) a tracked object based on the displayed view from the visible light detector and the generated outline.

### RELATED APPLICATIONS

This application claims priority benefit of U.S. Provisional Application Ser. No. 62/863,624 filed 19 Jun. 2019, the contents of which are hereby incorporated by reference.

### TECHNICAL FIELD

The present invention generally relates to optical tracking systems, and more particularly to a system and method to assist a user in positioning the field-of-view of an optical tracking system during a computer-assisted surgical procedure.

### BACKGROUND

Computer-assisted surgery is an expanding field having applications in total joint arthroplasty (TJA), bone fracture repair, maxillofacial reconstruction, and spinal reconstruction. Computer-assisted orthopedic surgical systems currently in field include the RIO® Robotic Arm Interactive Orthopedic System (Stryker-Mako, Kalamazoo, Mich.), the Navio™ Surgical System (Smith & Nephew, London, United Kingdom), and the ROSA® Robotic System (Zimmer-Biomet, Warsaw, Ind.). Each system utilizes a robotic device and an optical tracking system to help prepare the bone to receive an implant in a planned position and orientation (POSE). Optical tracking systems ensure the bone is prepared as planned by tracking the position of the robotic device relative to the patient's anatomy. Optical tracking systems are a key component to many computer-assisted surgical systems and are widely used in the operating room (OR).

With reference to FIG. 1, a particular example of a prior-art computer-assisted surgical system 10 with an optical tracking system 12 is shown in the context of an operating room. The computer-assisted surgical system 10 includes an optical tracking system 12, a tracked hand-held surgical device 14, and a display 16. The hand-held surgical device 14 includes an end-effector 15 that is actuated in two degrees-of-freedom to assist in creating one or more planar bone cuts during a total knee arthroplasty (TKA) procedure as further described in U.S. Patent Publication No. 2018/0344409 assigned to the assigned of the present application and incorporated by reference herein in its entirety. The display 16 displays information relative to the surgical procedure such as workflow instructions, prompts, patient information, device data, and may further temporarily display a field-of-view of the optical tracking system 12 as described below.

The optical tracking system 12 includes two or more optical detectors (18a, 18b) (e.g., optical cameras), and one or more processors to track the position and orientation (POSE) of objects in the field-of-view (FOV) of the optical detectors (18a, 18b) as further described in U.S. Pat. No. 6,601,644 incorporated by reference herein in its entirety. The optical detectors (18a, 18b) may be attached to the outside or integrated inside a surgical lamp 22 for an optimal viewing angle. In general, the optical detectors (18a, 18b) detect light emitted or reflected from three or more fiducial markers (e.g., active light emitting diode (LED), a retroreflective sphere) arranged on a rigid body or directly integrated onto a tracked device. Fiducial markers arranged on a rigid body are collectively referred to as a tracking array (20a, 20b, 20c), where each tracking array 20 has a unique arrangement of fiducial markers or a unique transmitting wavelength/frequency to permit the tracking system 12 to differentiate between the different objects being tracked. To differentiate the fiducial markers from background objects, the optical detectors (18a, 18b) are configured to detect infrared light only by way of a filter or other mechanism. The fiducial markers likewise reflect or emit infrared light. This allows the processor to pinpoint and triangulate the position of each fiducial marker without visible light interference.

As shown in FIG. 1, a tibia T, a femur F, and the hand-held surgical device 14 are tracked via a first tracking array 20a assembled to the tibia T, a second tracking array 20b assembled to the femur F, and a third tracking array 20c integrated with the hand-held surgical device 14. To accurately track each of these objects (e.g., femur F, tibia T, surgical device 14) during a surgical procedure, it is imperative that at least three fiducial markers on each tracking array 20 are within the FOV of the optical detectors (18a, 18b). To assist a user in positioning the optical detectors (18a, 18b), the view from the optical detectors (18a, 18b) may be displayed on the display 16 while a user adjusts the position of the optical detectors (18a, 18b). However, since the optical detectors (18a, 18b) are tuned to detect infrared light only, the fiducial markers are the only things visible on the display 16 as shown in FIG. 1, where each black dot 24 represents a fiducial marker and each cluster of black dots represents a tracking array (20a, 20b, 20c). With this limited information, it can be difficult for a user to aim the optical detectors (18a, 18b) in the correct spot. In addition, there are other relevant items that should be in the FOV (e.g., the patient, the surgical site) that are not visible in the infrared spectrum and may be pertinent to the future positions of the tracked objects during the procedure.

Thus, there exists a need for a system and method to assist a user in optimizing the FOV of an optical tracking system during a computer-assisted surgical procedure that accounts for additional relevant items in the OR invisible to an infrared optical tracking system

### SUMMARY OF THE INVENTION

A method is provided to assist in positioning the field-of-view (FOV) of an optical tracking system during a computer-assisted surgical procedure. The method includes displaying a view from a visible light detector on a display, and generating an outline as an overlay on the display of a FOV of two or more optical tracking detectors on the displayed view from the visible light detector. A user then positions at least one of: a) the two or more optical tracking detectors, or b) a tracked object based on the displayed view from the visible light detector and the generated outline.

A computer-assisted surgical system is provided. The system includes a tracking system with a visible light detector and two or more optical tracking detectors, one or more processors, and a display. The one or more processors execute software, and are in communication with or part of the tracking system which tracks positions of a set of fiducial markers. The display is used for displaying a view from the visible light detector, where the software when executed by the processor causes the processor to generate an outline as an overlay on the display of a FOV of the two or more optical tracking detectors on the displayed view from the visible light detector.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 is an example of a prior-art computer-assisted surgical system with an optical tracking system that is shown in the context of an operating room;

FIG. 2A depicts an optical tracking system attached to a surgical lamp in accordance with an embodiment of the invention;

FIG. 2B depicts an optical tracking system attached to a stand in accordance with an embodiment of the invention;

FIG. 3A illustrates a display that is displaying the view from the visible light detector positioned on a surgical lamp in accordance with an embodiment of the invention;

FIG. 3B illustrates the display of FIG. 3A that is displaying a variable change in size of the optical tracking detector FOV that changes based on the distance of a tracked object from the optical detectors in accordance with an embodiment of the invention;

FIG. 4 depicts a surgical system in the context of an operating room (OR) with a hand-held surgical device for use with the optical tracking system of FIG. 2A in accordance with an embodiment of the invention; and

FIG. 5 depicts a surgical system in the context of an operating room (OR) with a surgical robot for use with novel optical tracking system of FIG. 2B in accordance with an embodiment of the invention.

### DETAILED DESCRIPTION

The present invention has utility as a system and method to assist a user in optimizing the field-of-view (FOV) of an optical tracking system during a computer-assisted surgical procedure. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to computer-assisted orthopedic surgical procedures, the systems and methods may be applied to other medical and non-medical applications. However, a surgical setting is particularly apt for the present invention due to the limited space in the operating room (OR) (less room for error when positioning the optical detectors), and the clinical and technical considerations required for computer-assisted surgery.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “real-time” refers to the processing of input data within milliseconds such that calculated values are available within 2 seconds of computational initiation.

As used herein, the term “digitizer” refers to a measuring device capable of measuring physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording of physical points in space with a digitizer.

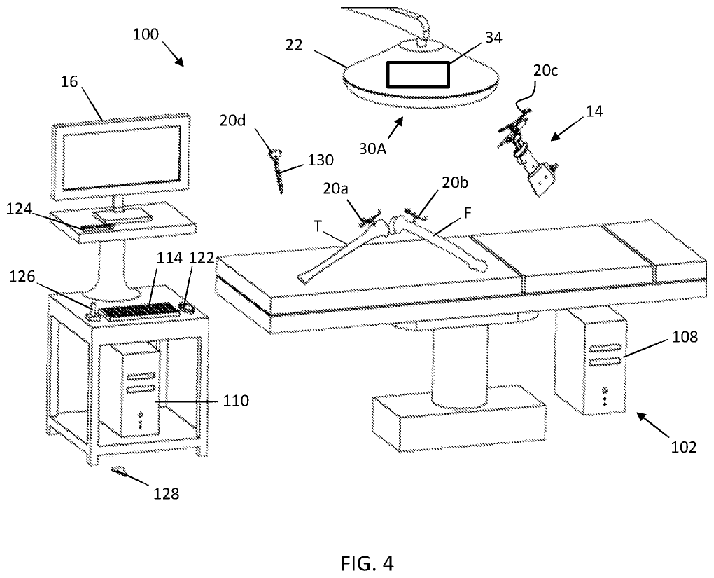

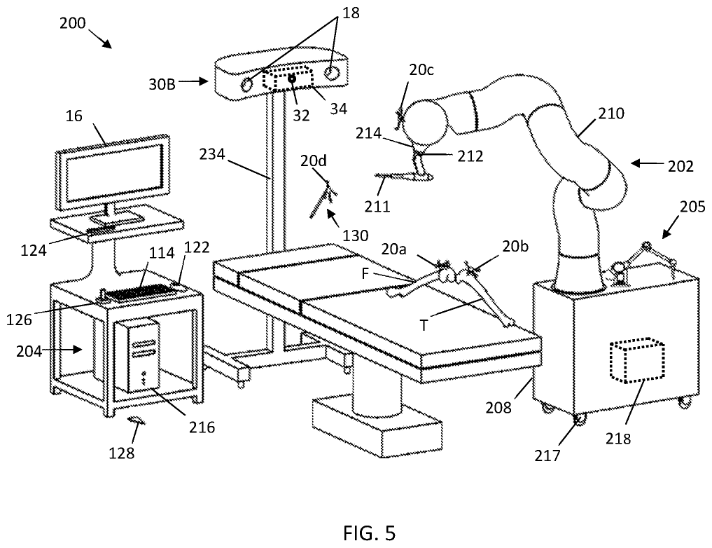

Also described herein are “computer-assisted surgical systems.” A computer assisted surgical system refers to any system requiring a computer to aid in a surgical procedure. Examples of computer-assisted surgical systems include 1-N degree of freedom hand-held surgical systems, tracking systems, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401; 7,206,626; 8,876,830; 8,961,536; and 9,707,043; and PCT Publication WO2017/058620. A robotic surgical system may provide active/automatic control, semi-active/semi-automatic control, haptic control, power control, or any combination thereof. Examples of specific surgical systems are described below with reference to FIGS. 4 and 5.

Also, referenced herein is a surgical plan. For context, the surgical plan is created, either pre-operatively or intra-operatively, by a user using planning software. The planning software may be used to generate three-dimensional (3-D) models of the patient's bony anatomy from a computed tomography (CT), magnetic resonance imaging (MRI), x-ray, ultrasound image data set, or from a set of points collected on the bone intra-operatively. A set of 3-D computer aided design (CAD) models of the manufacturer's prosthesis are pre-loaded in the software that allows the user to place the components of a desired prosthesis to the 3-D model of the boney anatomy to designate the best fit, position, and orientation of the implant to the bone.

Also used herein is the term “optical communication” which refers to wireless data transfer via infrared or visible light as described in U.S. Pat. No. 10,507,063 assigned to the assignee of the present application and incorporated by reference herein in its entirety.

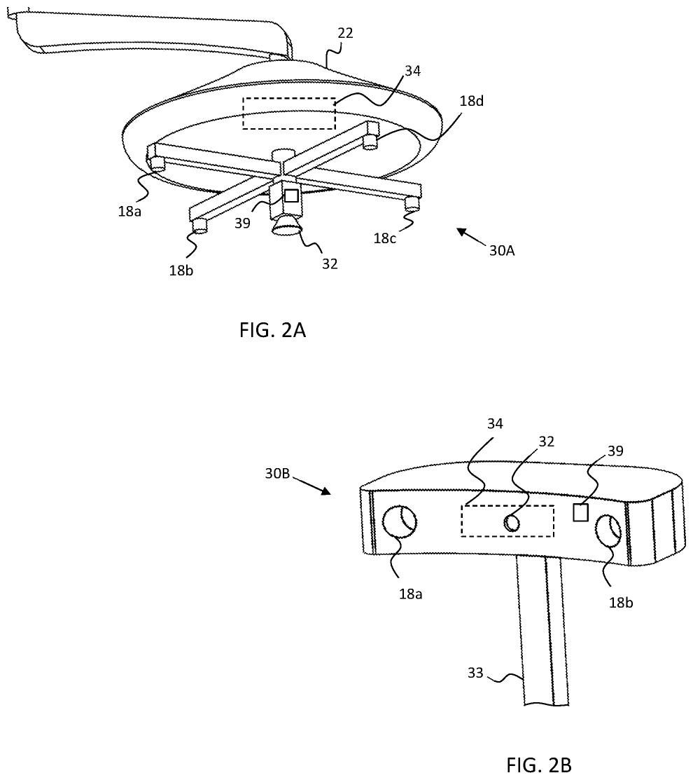

With reference now to the drawings, FIGS. 2A and 2B depict embodiments of a novel optical tracking system (30A, 30B) to assist a user in optimizing the FOV of the optical tracking system (30A, 30B), where FIG. 2A depicts the novel optical tracking system 30A attached to a surgical lamp 22, and FIG. 2B depicts the novel optical tracking system 30B attached to a stand 33. Embodiments of the novel optical tracking system (30A, 30B) include two or more optical tracking detectors (18a, 18b, 18c, 18d) (four detectors shown in FIG. 2A and two detectors shown in FIG. 2B), at least one visible light detector 32, and one or more tracking computers 34. The optical tracking detectors (18a, 18b, 18c, 18d) are configured to detect infrared light emitted or reflected from fiducial markers attached to a tracked object. The optical tracking detectors (18a, 18b, 18c, 18d) may be CCD cameras, CMOS cameras, optical scanners, or other light-sensing devices tuned to detect infrared light by way of a filter, embedded software, or other techniques known in the art. The visible light detector 32 is fixed into position relative to the optical tracking detectors (18a, 18b, 18c, 18d) such that the FOV of the visible light detector 32 can exceed the FOV of the optical tracking detectors (18a, 18b, 18c, 18d) as further described below. The visible light detector 32 may be a charged coupled device (CCD) camera, complementary metal-oxide-semiconductor (CMOS) camera, or other light-sensing device that detects visible light. As used herein, infrared light refers to electromagnetic radiation having a wavelength range anywhere between 700 nanometers to 1 millimeter, and visible light refers to electromagnetic radiation having a wavelength range anywhere between 380 nanometers to 740 nanometers. The one or more tracking computers 34 include hardware (e.g., processor(s), non-volatile memory, and/or controllers) and software to detect the POSE of fiducial markers, tracking arrays, and/or objects in 3-D space. Methods of tracking an object with two or more optical detectors and a processor are known in the art, such as the tracking system described U.S. Pat. No. 6,601,644.

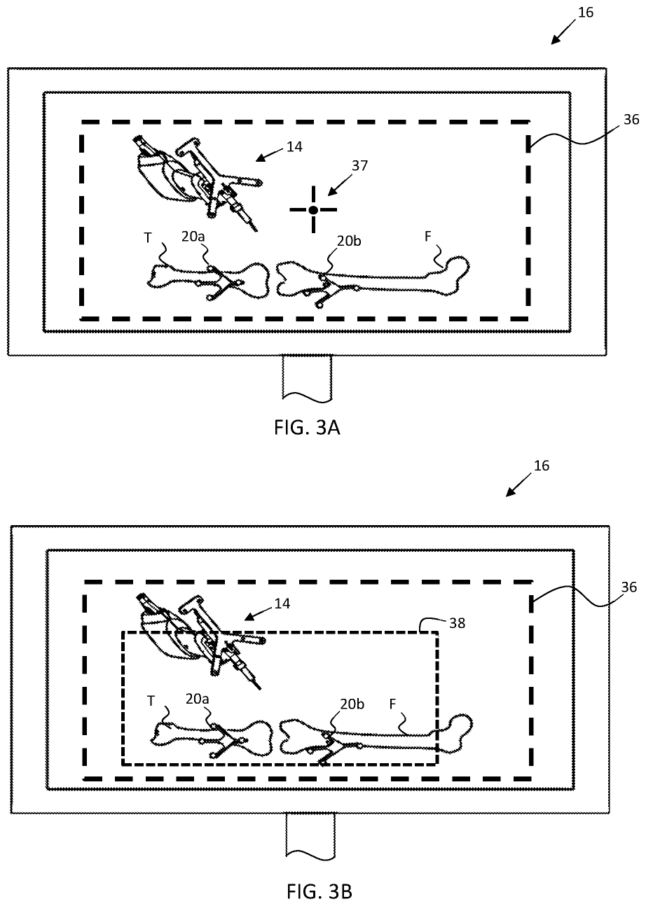

A method to assist a user in optimizing the FOV of embodiments the novel optical tracking system (30A, 30B) will now be described with the aid of FIGS. 3A and 3B. FIG. 3A illustrates a display 16 displaying the view from the visible light detector 32. Here, the visible light detector 32 is positioned on a surgical lamp 22 above an operating table with the visible light detector capturing the surgical device 14, the tibia T, and the femur F therein. One or more processors or computers (e.g., tracking computer 34, or a device computer as described with reference to FIG. 4 or 5) executing control software causes the display 16 to overlay an outline 36 of the optical tracking detectors FOV on the displayed view from the visible light detector 32. The outline 36 of the optical tracking detector FOV may be in the form of a bounded geometrical shape (e.g., rectangle, circle, oval), a semi-translucent shaded region, a bounded region filled with a gradient pattern, or other forms capable of indicating the optical tracking detectors FOV. The one or more processors may further cause the display 16 to overlay a marking 37 that indicates the center of the optical tracking detector FOV. The marking 37 may be in the form of cross-hairs, a diamond, a circle, or other geometric shapes that is overlaid on the displayed view from the visible light detector 32. Calibration techniques known in the art may be executed prior to the surgical procedure to ensure the optical tracking detector FOV is accurately depicted on the displayed view from the visible light detector 32. The position of the visible light detector 32 and optical tracking detectors (18a, 18b, 18c, 18d) may be fixed in relation to one another to maintain the accuracy of the system. In the OR, the displayed outline 36 reflects the optical tracking detectors FOV as a user adjusts the position of the optical tracking detectors (18a, 18b, 18c, 18d). This allows the user to optimize the position of the optical tracking detectors FOV and to account for additional objects (e.g., the surgical site, the patient) in the OR that are invisible to the optical tracking detectors (18a, 18b, 18c, 18d).

A method of using embodiments of the novel optical tracking system (30A, 30B) may include the following steps. The optical tracking detectors (18a, 18b, 18c, 18d) and the visual light detector 32 are positioned at a first location to visualize one or more tracked objects in the operating room. One or more processors cause a display to output the view from visual light detector 32 with an outline 36 of the optical tracking detectors FOV. The displayed outline 36 reflects the optical tracking detector FOV as a user adjusts the position of the two or more optical tracking detectors (18a, 18b, 18c, 18d). This assists the user in determining a location for the optical tracking detectors (18a, 18b, 18c, 18d) that optimizes the position of the optical tracking detector FOV. The surgical procedure begins with the optical tracking detectors (18a, 18b, 18c, 18d) at the optimized location. At any point during the procedure, the user may re-adjust the position of the optical tracking detectors (18a, 18b, 18c, 18d) using the displayed outline 36 to re-position the optical tracking detectors FOV.

In another embodiment, the user may adjust the position of any tracked objects relative to the position of the two or more optical tracking detectors (18a, 18b, 18c, 18d). The user may use the displayed outline 36 to move or position one or more tracked objects (e.g., tracked surgical device, tracked bones) relative to the displayed outline 36 while the position of the two or more optical tracking detectors (18a, 18b, 18c, 18d) remains unchanged. In a further embodiment, the user may adjust both the position of the two or more optical detectors and any tracked objects to optimize their positions relative to one another using the displayed outline 36 as a guide.

With reference to FIG. 3B, the novel optical tracking system (30A, 30B) may further account for the variable change in size of the optical tracking detector FOV that changes based on the distance of a tracked object from the optical detectors (18a, 18b, 18c, 18d). When using an optical tracking system (stereoscopic or multi-detectors), the FOV of the optical tracking detectors (18a, 18b, 18c, 18d) may change size in-plane depending on how far away the tracked object of interest is from the optical tracking detectors (18a, 18b, 18c, 18d). For example, the optical tracking detectors FOV may be greater for tracked objects closer to the optical tracking detectors (18a, 18b, 18c, 18d) compared to tracked objects farther from the optical detectors (18a, 18b, 18c, 18d). This change in size may be the result of the optical tracking detectors (18a, 18b, 18c, 18d) focusing back-and-forth between the different objects being tracked. To account for this variable change in size, the novel optical tracking system (30A, 30B) may execute one or more of the following. In a particular inventive embodiment, a single outline 36 of the optical tracking detectors FOV is displayed on the display 16 where the single outline 36 reflects the optical tracking detectors FOV for the closest tracked object to the optical detectors 18. The optical tracking system (30A, 30B) knows the position/depth of the closest tracked object and may therefore adjust the single outline 36 accordingly. In another inventive embodiments, multiple outlines (36, 38) may be displayed on the display 16 where each outline (36, 38) reflects the optical tracking detectors FOV for each of the tracked objects. For example, with reference to FIG. 3B, a first outline 36 may reflect the optical tracking detectors FOV for the tracked surgical device 14, while a second outline 38 reflects the optical tracking detectors FOV for the femur F and tibia T. Each outline (36, 38) therefore corresponds to the depth of another tracked object in the optical tracking detectors FOV. Each outline (36, 38) may have different indicia (e.g., a color or pattern) to differentiate the outlines (36, 38) from one another. Furthermore, each outline (36, 38) may have indicia or a label that matches to its tracked object or the tracking array associated with that tracked object. For example, the first outline 36 may be colored blue that matches with a blue colored tracking array integrated with the surgical device 14. The second outline 38 may be colored yellow that matches with a yellow colored tracking array attached to the femur F, and so on.

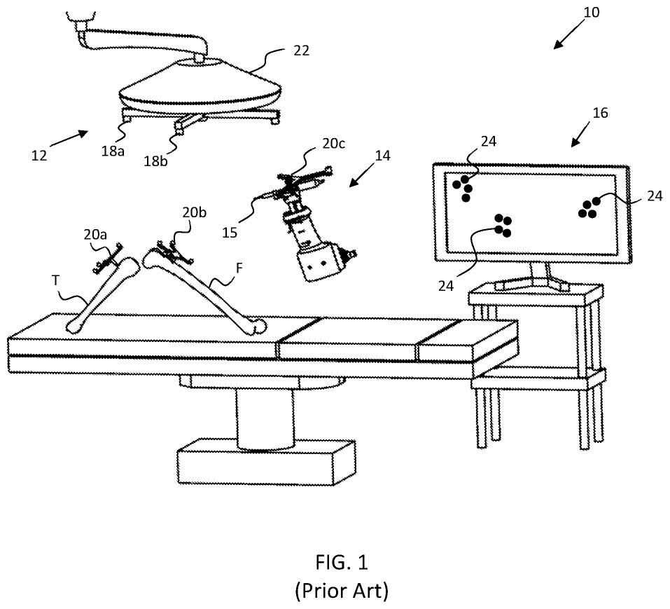

Another problem may arise while positioning the optical tracking detectors (18a, 18b, 18c, 18d). It is contemplated that the actual markers on the tracking array may be difficult to visualize on the displayed view from the visible light detectors. Therefore, in specific inventive embodiments, a virtual outline or indication of the actual markers may be displayed in the view from the visible light detector. For example, the position of the markers as depicted in FIG. 1 may be overlaid on the view from the visible light detector 32. This provides the user with an exact view of the markers in the FOV of the visible light detector 32. The virtual outline or indication of the actual markers may be displayed in conjunction with or absent to the display of one or more outlines (36, 38) of the optical tracking detectors FOV.

In a specific inventive embodiment, with reference back to FIGS. 2A and 2B, the novel optical tracking system (30A, 30B) may further include at least one motion detection device 39. The motion detection device 39 is configured to detect any motion of the two or more optical tracking detectors (18a, 18b, 18c, 18d). The motion detection device 39 is further configured to signal to the control software when the optical tracking detectors (18a, 18b, 18c, 18d) are moving and not moving, so that the control software, in response, can cause the display to automatically go into and out of an adjustment mode and a non-adjustment mode. For example, in response to the motion detection device 39 detecting motion of the optical tracking detectors (18a, 18b, 18c, 18d) (e.g., because a user is adjusting the position of the optical tracking detectors (18a, 18b, 18c, 18d)), the control software may automatically cause the display 16 to display the view from the visible light camera 32 and generate the overlay of the outline 36. This assists the user in positioning the optical tracking detectors (18a, 18b, 18c, 18d) (i.e., an adjustment mode). Once the motion detection device 39 no longer senses motion, the control software may go out of the adjustment mode causing the display 16 to display something other than the view from the visible light camera 32 and/or outline 36.

The motion detection device 39 may illustratively be an accelerometer, gyroscope, inertial measuring unit (IMU), strain gauge, or a second optical tracking system. The motion detection device(s) 39 may be attached or integrated with a surgical lamp or stand, or attached or integrated with an optical tracking detector (18a, 18b, 18c, 18d). It should be appreciated however that several other locations for the motion detection device 39 may exist that permits the motion detection device 39 to detect any motion of the two or more optical tracking detectors (18a, 18b, 18c, 18d). The motion detection device 39 is further in wired or wireless communication with the one or more aforementioned processors or computers executing the control software.

### Surgical Systems

FIG. 4 depicts a surgical system 100 in the context of an operating room (OR) with a hand-held surgical device 14 for use with the novel optical tracking system 30A described herein. FIG. 5 depicts a surgical system 200 in the context of an operating room (OR) with a surgical robot 202 for use with novel optical tracking system 30B described herein. The systems shown in FIGS. 4 and 5 will be described in a single discussion with common elements having the same reference number.

The surgical system 100 of FIG. 4 is described in more detail in U.S. Patent Publication No. 2018/0344409 assigned to the assignee of the present application. The 2-DOF surgical system 100 generally includes a computing system 102, a hand-held articulating surgical device 14 with a tracking array 20c, and the inventive embodiment of the optical tracking system 30A. The surgical system 100 is able to guide and assist a user in accurately placing pins or creating cuts on a bone for orthopedic surgery.

The computing system 102 may include: a navigation computer 108 including a processor; a planning computer 110 including a processor; a tracking computer 34 including a processor, and peripheral devices. Processors operate in the computing system 102 to perform computations associated with the inventive system and method. It is appreciated that processor functions may be shared between computers 108, 110, 34, or a subset thereof; a remote server; a cloud computing facility; or combinations thereof.

In particular inventive embodiments, the navigation computer 108 may include one or more processors, controllers, software, data, and data storage medium(s) such as RAM, ROM or other non-volatile or volatile memory to perform functions related to the surgical procedure. These functions illustratively include at least one of: controlling a surgical workflow; providing guidance to the user; interpreting pre-operative planning surgical data; and controlling the operation of the surgical device 14. In some embodiments, the navigation computer 108 is in direct communication with the optical tracking system 30A such that the optical tracking system 106 may identify trackable devices in the field of view (FOV) and the navigation computer 108 can control the workflow and/or control the surgical device 14 accordingly based on the identity and POSE of the tracked objects (e.g., surgical device 14, femur F, tibia T). In some embodiments, the navigation computer 108 is housed in the hand-held portion of the hand-held surgical device 14 to provide local control to the surgical device 14. The novel optical tracking system 30A may communicate information data, tracking data, and/or operational data to the navigation computer 108 via a wired or wireless connection. The wireless connection may be via visible light communication as described in U.S. Pat. No. 10,507,063 assigned to the assignee of the present application and incorporated by reference herein in its entirety. Furthermore, the navigation computer 108 and the tracking computer 34 may be separate entities as shown, or it is contemplated that their operations may be executed on just one or two computers depending on the configuration of the surgical system 100. For example, the tracking computer 34 may have operational data to directly control the workflow without the need for a navigation computer 108. Or, the navigation computer 108 may include operational data or control software to directly read data detected from the optical tracking detectors (18a, 18b, 18c, 18d) and/or cause the display 16 to display the view from the visible light detector 32 and generate the outline 36 without the need for a tracking computer 34.

The peripheral devices allow a user to interface with the surgical system 100 and may include: one or more user interfaces, such as a display or monitor 16; and various user input mechanisms, illustratively including a keyboard 114, mouse 122, pendent 124, joystick 126, foot pedal 128, or the monitor 16 may have touchscreen capabilities.

The planning computer 110 is preferably dedicated to planning the procedure either pre-operatively or intra-operatively. For example, the planning computer 110 may contain hardware (e.g. processors, controllers, and non-volatile memory), software, data, and utilities capable of receiving and reading medical imaging data, segmenting imaging data, constructing and manipulating three-dimensional (3D) virtual models, storing and providing computer-aided design (CAD) files, generating the surgical plan data for use with the system 100, and providing other various functions to aid a user in planning the surgical procedure. The final surgical plan data may include an image data set of the bone, bone registration data, subject identification information, the POSE of the implants relative to the bone, the POSE of one or more target planes defined relative to the bone, and any tissue modification instructions. The final surgical plan is readily transferred to the navigation computer 108 and/or tracking computer 34 through a wired or wireless connection in the operating room (OR); or transferred via a non-transient data storage medium (e.g. a compact disc (CD), a portable universal serial bus (USB drive)) if the planning computer 110 is located outside the OR.

The surgical system 100 further includes the novel optical tracking system 30A as described above. The novel optical tracking system 30A assists a user in optimizing the position of the FOV of the optical tracking cameras (18a, 18b, 18c, 18d) and to accurately track the hand-held surgical device 14, the femur F, and the tibia T during the surgical procedure. The tracking system computer 34 includes tracking hardware, software, data, and utilities to determine the POSE of objects (e.g., bones such as the femur F and tibia T, the surgical device 14) in a local or global coordinate frame. The POSE of the objects is referred to herein as POSE data or tracking data, where this POSE data is readily communicated to the navigation computer 108. The tracking system computer 34 is in wired or wireless communication with the display monitor 16 to cause the display monitor 16 to display an overlay 36 of the FOV of the optical tracking detectors 18 on the displayed view from the visible light detector 32 as shown in FIGS. 3A and 3B.

The surgical system 100 further includes a tracked digitizer probe 130. The digitizer probe 130 is tracked via a tracking array 20d attached or integrated with the tracked digitizer probe 130. The tracked digitizer probe 130 aids in the collection, measurement, or recordation of points in 3-D space. The collection of points may be used to facilitate the registration of the bones to a surgical plan.

Referring now to surgical system 200 of FIG. 5, in which like numbered aspects have the meaning ascribed thereto with respect to the aforementioned figures, the surgical robot 202 may include a movable base 208, a manipulator arm 210 connected to the base 208, an end-effector 211 located at a distal end 212 of the manipulator arm 210, and a force sensor 214 positioned proximal to the end-effector 211 for sensing forces experienced on the end-effector 211. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 211 in various degrees of freedom. The joints are illustratively prismatic, revolute, spherical, or a combination thereof. The surgical robot 202 further includes a tracking array 20c to track the position of the end-effector 211. The tracking array 20c may be attached to the end-effector 211 to track the end-effector 211 directly, or the tracking array 20c may be positioned on the base 208 or a link of the surgical robot 202 where the kinematics of the surgical robot is used with the tracking data to track the POSE of the end-effector 211.

The computing system 204 generally includes a planning computer 216; a device computer 218; a tracking computer 34; and peripheral devices. The planning computer 216, device computer 218, and tracking computer 34 may be separate entities, one-in-the-same, or combinations thereof depending on the surgical system. Further, in some embodiments, a combination of the planning computer 216, the device computer 218, and/or tracking computer 34 are connected via a wired or wireless communication. The peripheral devices allow a user to interface with the surgical system components and may include: one or more user-interfaces, such as a display or monitor 16; and user-input mechanisms, such as a keyboard 114, mouse 122, pendent 124, joystick 126, foot pedal 128, or the monitor 16 that in some inventive embodiments has touchscreen capabilities.

The planning computer 216 contains hardware (e.g., processors, controllers, and/or memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include pre-operative bone data, patient data, registration data including the POSE of a set of points P defined relative to the pre-operative bone data, and/or operational data. The operational data may be a set of instructions for modifying a volume of tissue that is defined relative to the anatomy, such as a set of cutting parameters (e.g., cut paths, velocities) in a cut-file to autonomously modify the volume of bone, a set of virtual boundaries defined to haptically constrain a tool within the defined boundaries to modify the bone, a set of planes or drill holes to drill pins or tunnels in the bone, or a graphically navigated set of instructions for modifying the tissue. In particular embodiments, the operational data specifically includes a cut-file for execution by a surgical robot to automatically modify the volume of bone, which is advantageous from an accuracy and usability perspective. The surgical plan data generated from the planning computer 216 may be transferred to the device computer 218 and/or tracking computer 34 through a wired or wireless connection in the operating room (OR); or transferred via a non-transient data storage medium (e.g., a compact disc (CD), a portable universal serial bus (USB) drive) if the planning computer 216 is located outside the OR. In specific embodiments, the wireless communication of the surgical planning data to the device computer 218 is accomplished via visible light communication.

The device computer 218 in some inventive embodiments is housed in the moveable base 208 and contains hardware, software, data and utilities that are preferably dedicated to the operation of the surgical robotic device 202. This may include surgical device control, robotic manipulator control, the processing of kinematic and inverse kinematic data, the execution of registration algorithms, the execution of calibration routines, the execution of operational data (e.g., cut-files, haptic constraints), coordinate transformation processing, providing workflow instructions to a user, and utilizing position and orientation (POSE) data from the tracking system 30B. In some embodiments, the surgical system 200 includes a mechanical digitizer arm 205 attached to the base 208. The digitizer arm 205 may have its own digitizer computer or may be directly connected with the device computer 218. The mechanical digitizer arm 205 may act as a digitizer probe that is assembled to a distal end of the mechanical digitizer arm 205. In other inventive embodiments, the system includes a tracked digitizer probe 130 with a probe tip and a tracking array 20d.

The surgical system 100 further includes the novel optical tracking system 30B as described above. The novel optical tracking system 30B assists a user in optimizing the position of the FOV of the optical tracking cameras 18 to accurately track the surgical robot 202, the femur F, and the tibia T during the surgical procedure. The tracking system computer 34 includes tracking hardware, software, data, and utilities to determine the POSE of objects (e.g., bones such as the femur F and tibia T, end-effector 211 of the surgical robotic device 202) in a local or global coordinate frame. The POSE of the objects is referred to herein as POSE data or tracking data, where this POSE data is readily communicated to the device computer 218. The tracking system computer 34 is in wired or wireless communication with the display 16 to cause the display 16 to display an overlay 36 of the FOV of the optical tracking detectors 18 in the displayed view from the visible light detector 32.

POSE data or tracking data is determined by the novel optical tracking system 30B using the position data detected from the optical tracking detectors 18 and operations/processes such as image processing, image filtering, triangulation algorithms, geometric relationship processing, registration algorithms, calibration algorithms, and coordinate transformation processing.

The POSE data is used by the computing system 204 during the procedure to update the POSE and/or coordinate transforms of the bone B, the surgical plan, and the surgical robot 202 as the manipulator arm 210 and/or bone(s) (F, T) move during the procedure, such that the surgical robot 202 can accurately execute the surgical plan.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method to assist in positioning one or more optical detectors of an optical tracking system, said method comprising:
displaying a view from a visible light detector on a display; and
displaying a first field-of-view (FOV) of the one or more optical detectors on the displayed view.

2. The method of claim 1 wherein the first FOV of the one or more optical detectors is an outline overlaid on the displayed view.

3. The method of claim 1 wherein said one or more optical detectors are configured to detect infrared light.

4. The method of claim 1 wherein said visible light detector is in a fixed position relative to said one or more optical detectors.

5. The method of claim 2 wherein the outline comprises bounded geometrical shape, a semi-translucent shaded region, or a bounded region filled with a gradient pattern.

6. The method of claim 1 further comprising displaying a marking indicating a center of the first FOV of the one or more optical tracking detectors on the displayed view.

7. (canceled)

8. The method of claim 21 further comprising displaying two or more fields-of-view (FOVs) of said two or more optical detectors on the displayed view, wherein each of said two or more FOVs correspond to a second FOV of said two or more optical detectors when tracking an object.

9. The method of claim 8 further comprising automatically updating each of said two or more FOVs based on a distance of the object relative to the two or more optical detectors.

10. (canceled)

11. The method claim 8 wherein each of said two or more FOVs are displayed as on outline of the second FOV of the two or more optical detectors.

12. The method of claim 8 wherein each of said two or more FOVs are differentiated from one another by at least one of: an indicia, a color, or a label.

13. The method of claim 1 further comprising displaying a position of fiducial markers in the first FOV on the displayed view.

14. The method of claim 3 wherein said optical detector is a camera.

15. (canceled)

16. A system, comprising:
an optical tracking system comprising one or more optical detectors;
a visible light detector in a fixed position with respect to the or more optical detectors; and
one or more processors executing software, wherein said software when executed by the processor
displays a first field-of-view (FOV) of said one or more optical detectors on a displayed view from the visible light detector.

17. (canceled)

18. The system of claim 16 wherein said one or more optical detectors are configured to detect infrared light.

19. The system of claim 16 further comprising a hand-held surgical device or a surgical robot.

20. The system of claim 16 wherein the software when executed by the one or more processors displays a marking on the displayed view, wherein the marking indicates a center of the first FOV of said one or more optical detectors.

21. The method of claim 1 wherein the optical tracking system comprises two or more optical detectors.

22. The system of claim 16 wherein the FOV of said optical detector is displayed as an outline of said one or more optical detectors overlaid on the displayed view.

23. The system of claim 16 wherein the optical tracking system comprises two or more optical detectors.

24. The system of claim 16 wherein the optical tracking system comprises the one or more processors and software.
